(19) United States
(12) Patent Application Publication
(10) Pub. No.: US 2026/0094705 A1
(43) Pub. Date: Apr. 2, 2026

(54) SYSTEMS AND METHODS FOR COMPUTER VISION FAULT DETECTION IN DIALYSIS APPARATUS

(71) Applicant: MOZARC MEDICAL US LLC, Minneapolis, MN (US)

(72) Inventors: Murugavel SWAMINATHAN, Thanjavur (IN); Bindhu ANJANEYA, Bangalore (IN); Sharath NAGENDRA, Bangalore (IN)

(73) Assignee: MOZARC MEDICAL US LLC, Minneapolis, MN (US)

(21) Appl. No.: 19/251,077

(22) Filed: Jun. 26, 2025

(30) Foreign Application Priority Data
Jun. 27, 2024 (IN) 202411049374

(51) Int. Cl. G16H 40/40; G16H 40/60

(52) U.S. Cl. CPC G16H 40/40; G16H 40/60

(57) ABSTRACT

Systems and methods of the present disclosure enable fault risk detection in renal care systems and/or devices, by receiving, from an image sensor, an image of a component of the renal care system/device and using a fault risk detection machine learning model to output a detected fault risk in the component based at least in part on trained machine learning model parameters configured, through training, to classify the detected fault risk as matching to one or more known fault risks. An alert is generated to an operator or technician of the renal care system/device, including an indication of the detected fault risk.

### CROSS REFERENCE TO RELATED APPLICATION

This application claims priority to Indian Provisional Application 202411049374, filed Jun. 27, 2024 and titled “SYSTEMS AND METHODS FOR COMPUTER VISION FAULT DETECTION IN DIALYSIS APPARATUS” which is incorporated herein by reference in its entirety.

### FIELD OF TECHNOLOGY

The present disclosure generally relates to computer-based systems and methods for utilizing machine learning (ML) based computer vision to automatically detect faults and/or fault risks in apparatuses such as dialysis and other medical treatment apparatuses.

### BACKGROUND OF TECHNOLOGY

Dialysis is a treatment that helps remove waste products and excess fluids from the blood when the patient's organ, such as kidneys, are unable to perform this function effectively. To do so, dialysis filters blood extracorporeally using one or more filters, membranes, dialysates, and other components and materials. Thus, dialysis may performs some of the kidney's functions, such as removing waste and extra fluids from the blood in order to helps regulate minerals (like potassium, sodium, calcium, and bicarbonate) and blood pressure.

### SUMMARY

In some aspects, the techniques described herein relate to a method including: receiving, by at least one processor from at least one image sensor positioned on a renal care device, at least one image of at least one component of the renal care device; inputting, by the at least one processor, the at least one image into a fault risk detection machine learning model to output at least one detected fault risk in the at least one component based at least in part on trained machine learning model parameters; wherein the trained machine learning model parameters are configured, through training, to classify the at least one detected fault risk as matching to at least one known fault risk; wherein the training includes: inputting a plurality of training images into the fault risk detection machine learning model, the plurality of training images including known fault risks to the at least one component of the renal care device, and updating a plurality of machine learning model parameters based at least in part on the plurality of training images and the known fault risks so as to produce the trained machine learning model parameters; and generating, by the at least one processor, at least one alert to at least one operator of the renal care device, the at least one alert including an indication of the at least one detected fault risk.

In some aspects, the techniques described herein relate to a system including: a renal care device including at least one image sensor, at least one component and at least one processor; wherein the at least one image sensor is positioned on the renal care device so as to capture at least one image of the at least one component; wherein the at least one processor is configured to: receive, from the at least one image sensor, the at least one image of the at least one component of the renal care device; input the at least one image into a fault risk detection machine learning model to output at least one detected fault risk in the at least one component based at least in part on trained machine learning model parameters; wherein the trained machine learning model parameters are configured, through training, to classify the at least one detected fault risk as matching to at least one known fault risk; wherein the training includes: inputting a plurality of training images into the fault risk detection machine learning model, the plurality of training images including known fault risks to the at least one component of the renal care device, and updating a plurality of machine learning model parameters based at least in part on the plurality of training images and the known fault risks so as to produce the trained machine learning model parameters; and generate at least one alert to at least one operator of the renal care device, the at least one alert including an indication of the at least one detected fault risk.

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

The term “comprising” includes, but is not limited to, whatever follows the word “comprising.” Use of the term indicates the listed elements are required or mandatory but that other elements are optional and may be present.

The terms “control,” “controlling,” or “controls” refer to the ability of one component to direct the actions of one or more second or other components.

A “control system” can be a device that monitors and affects the operational conditions of a given system. The operational conditions are typically referred to as output variables of the system wherein the output variables can be affected by adjusting certain input variables. The control system can be any number or combination of processors, controllers, software, and computers.

The terms “determining,” “determines,” and the like, generally refer to, in the broadest reasonable interpretation, any process or method for obtaining or coming to a decision, value, number, or finding, for any one or more value, output, parameter, or variable, by any means applicable to the relevant parameter being determined.

A “dialysis session” can be any time period of any length during which a patient is treated by or undergoes dialysis, hemodialysis, hemofiltration, ultrafiltration, or other fluid removal therapy.

The term “dialyzer” refers to a cartridge or container with two flow paths separated by semi-permeable membranes. One flow path is for blood and one flow path is for dialysate. The membranes can be in hollow fibers, flat sheets, or spiral wound or other conventional forms known to those of skill in the art. Membranes can be selected from the following materials of polysulfone, polyethersulfone, poly (methyl methacrylate), modified cellulose, or other materials known to those skilled in the art.

The term “downstream” refers to a position of a first component in a flow path relative to a second component wherein fluid will pass by the first component after the second component during normal operation. The first component can be said to be “downstream” of the second component, while the second component is “upstream” of the first component.

The term “fluidly connected” refers to a particular state or configuration of one or more components such that fluid, gas, or combination thereof, can flow from one point to another point. The connection state can also include an optional unconnected state or configuration, such that the two points are disconnected from each other to discontinue flow. It will be further understood that the two “fluidly connectable” points, as defined above, can form a “fluidly connected” state. The two points can be within or between any one or more of compartments, modules, systems, components, all of any type.

The terms “initiating” or to “initiate” a process refer to beginning a series of steps or operations.

The term “mode of operation” refers to the way a system or component operates. For example, a dialysis system can operate as a single pass system or a multi-pass system.

The term “monitoring” or to “monitor” refers to ascertaining a state of a system or process.

The term “multi-pass mode” refers to a mode of operating a dialysis system wherein at least a portion of spent dialysate is regenerated and pumped through a dialyzer multiple times during therapy.

A “patient” or “subject” can be a member of any animal species, preferably a mammalian species, optionally a human. The subject can be an apparently healthy individual, an individual suffering from a disease, or an individual being treated for a disease. In certain embodiments, the patient can be a human, sheep, goat, dog, cat, mouse, or any other animal.

A “patient parameter” is any data that gives relevant information about the health status and therapy requirements of a patient.

A “system parameter” is any data that gives relevant information about a system, including concentrations of fluids to be used by the system or data concerning one or more components of the system.

The term “upstream” refers to a position of a first component in a flow path relative to a second component wherein fluid will pass by the first component before the second component during normal operation. The first component can be said to be “upstream” of the second component, while the second component is “downstream” of the first component.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 illustrates an illustrative dialysis system in accordance with one or more embodiments.

FIG. 2 depicts a block diagram of an exemplary electronic control device(s) configured to detect a state of, and provide electronic instruction to components of the dialysis system in accordance with one or more embodiments of the present disclosure.

FIGS. 3A and 3B depict illustrative examples of fault risks for components in a dialysis system in accordance with one or more embodiments of the present disclosure.

FIG. 4 depicts an exemplary machine learning training methodology for training one or more image classifiers to detect fault risks in a dialysis system in accordance with one or more embodiments of the present disclosure.

FIG. 5 depicts an exemplary machine learning classification methodology for detecting fault risks in a dialysis system in accordance with one or more embodiments of the present disclosure.

FIG. 6 is a flow chart for a method of detecting fault risks in a dialysis system in accordance with one or more embodiments of the present disclosure.

FIG. 7 depicts a block diagram of another exemplary computer-based system and platform for detecting fault risks in a dialysis system in accordance with one or more embodiments of the present disclosure.

### DETAILED DESCRIPTION

Various detailed embodiments of the present disclosure, taken in conjunction with the accompanying FIGs., are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

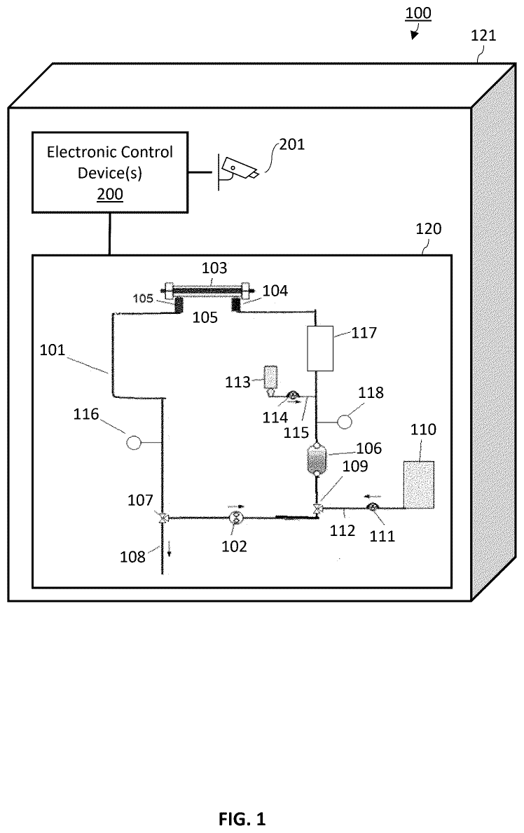

FIGS. 1 through 8 illustrate systems and methods of automated fault risk detection in renal care systems using ML-based computer vision. Renal care systems, such as dialysis machines and components thereof, include a variety of components including electronics, processing devices, valves, tubing, structural supports, and other components. The renal care system leverages such components to filter blood or other bodily fluids, often using dialysates, sorbents and other chemicals and materials. However, such chemicals and materials can be a cause of corrosion to various of the components, may leave deposits of material that may effect the operation of components, such as blockages to tubes and/or valves or to mechanical actuators. Indeed, due to the temperature at which materials are kept, there may be additional condensation that can cause corrosion and other sources of faults or otherwise render the system and/or component(s) thereof inoperability. This is all in addition to general wear and tear, and mechanical sources of faults such as, e.g., vibration, impacts (e.g., due to the system being bumped or knocked over), breakdown of high wear areas, such as gaskets, bearings, couplings, fasteners, switches, etc. Accordingly, the renal care system may be susceptible to influences that can cause faults, such as, defects, wear, breakdown or other risks of rendering the renal care system inoperable. Such faults can be costly, inconvenient and dangerous to a patient that relies of the renal care system.

The following embodiments provide technical solutions and technical improvements that overcome technical problems, drawbacks and/or deficiencies in the technical fields involving the automated detection of risks of fault in a renal care system. The automated detection may be achieve via one or more imaging sensors positioned in, on and/or around the renal care system for visual monitoring of components of the renal care system such as an ML-based image processing pipeline may automatically detect conditions that are associated with a risk of fault. As a result, such conditions can be detected prior to and/or upon the occurrence of a fault so as to identify the source of a fault and/or risk thereof. As a result, the renal care system may be made more reliable and safe via the early identification of conditions that are associated with the occurrence of faults, and thus improving patient care.

Based on such technical features, further technical benefits become available to users and operators of these systems and methods. Moreover, various practical applications of the disclosed technology are also described, which provide further practical benefits to users and operators that are also new and useful improvements in the art.

FIG. 1 illustrates a system for improving the operational capabilities of a renal care system 100. The renal care system 100 can include a fluid flow path 101 fluidly connectable to a dialyzer inlet 104 and a dialyzer outlet 105 of a dialyzer 103. Dialysate is pumped through a first side of the dialyzer 103, while blood from a patient is pumped through the opposite side. Solutes, toxins, and water can cross a dialyzer membrane (not shown) to pass from the blood into the dialysate. Pump 102 provides the driving force for moving dialysate through the fluid flow path 101.

In some embodiments, in a sorbent based multi-pass renal care system 100, spent dialysate is pumped through a sorbent cartridge 106. The sorbent cartridge 106 can contain sorbent materials, such as activated carbon, alumina and urease, zirconium phosphate, and zirconium oxide. These sorbent materials can remove waste solutes from the spent dialysate. The activated carbon in the sorbent cartridge 106 can remove non-ionic waste solutes, such as creatinine, glucose, uric acid, β2-microglobulin and other non-ionic toxins, except urea. Alternative sorbents can also be used that can remove urea directly.

Because the cation exchange material, such as zirconium phosphate, removes potassium, calcium and magnesium from the dialysate, these ions need to be added back into the dialysate before the dialysate can be used in dialysis. In some embodiments, adding ions or other solutes back into the dialysate can be accomplished by use of an infusate system. Infusate source 113 can contain concentrates or solids of each of the necessary cations, as well acid, sodium chloride and sodium bicarbonate. The necessary cations can be added to the regenerated dialysate from infusate source 113 through infusate line 115 by pump 114. In some embodiments, the infusates can be located in separate containers (not shown) each with dedicated pumps and need not be present in a single container as illustrated in FIG. 1. Further, other additives may be added to the regenerated dialysate in the same fashion, such as bicarbonate or acetate buffers.

In some embodiments, the zirconium phosphate or other cation exchange resin in the sorbent cartridge 106 can remove ammonium ions generated by catalytic breakdown of urea by the urease. The zirconium phosphate also acts as a buffer depending on pH of the zirconium phosphate and removes bicarbonate as needed to maintain a certain pH. Once the capacity of the zirconium phosphate has been reached, the generated ammonium ions will pass through the sorbent cartridge into the dialysate, forcing dialysis treatment to stop. Further, even before reaching capacity, the zirconium phosphate can pH titrate due to adsorption of ammonium and other cations and reaction with bicarbonate in the spent dialysate, causing release of sulfate ions adsorbed earlier in a dialysis session. An increasing pH can also increase the bicarbonate present in the sorbent cartridge effluent, which may contribute to errors in bicarbonate control. Additionally, sulfate and nitrate ions may be difficult to remove using a sorbent cartridge due to low capacity of the sorbent material.

In some embodiments,, valve 107 can be a proportioning valve. Valve 107 can be capable of diverting between 0%-100% of the flow to drain 108. Valve 107 can enable diversion of fraction between 0-100% to enable over dilution when renal care system 100 is using multi-pass mode. In certain embodiments, the mode of operation of the renal care system 100 can be between single pass and multi-pass mode. Valve 107 can be operated to divert a percentage of the dialysate to the drain, and additional water can be added. For example, 50% of the dialysate can be diverted to the drain, with a volume of water added to offset the volume of dialysate removed, rather than removing only 0 or 100% of the dialysate. Removing only a portion of the dialysate reduces the amount of dialysate that passes through the sorbent cartridge, increasing the length of time before capacity is reached, while using less water than purely single-pass mode.

In some embodiments, in the single pass mode, the spent dialysate is discarded through drain line 108, which can be fluidly connected to a drain or a waste container (not shown). Fresh water from water source 110 can be pumped into fluid flow path 101 through water line 112. Pump 111 can provide the driving force for moving water through water line 112. In single pass mode, valve 109 can be opened to the water line 112 to allow water into fluid flow path 101.

In some embodiments, water source 110 can contain potable water, purified water, or pre-made dialysate. If potable water is used, the water source 110 can be upstream of the sorbent cartridge 106 to allowing the water to be purified before reaching the dialyzer. If water source 110 contains premade dialysate, the dialysate added to fluid flow path 101 can be added downstream of sorbent cartridge 106 and directly pumped through dialyzer 103. If water source 110 contains purified water, the water source 110 can be either upstream or downstream of the sorbent cartridge 106, and the infusate system can be used to generate dialysate in-line by adding the components of the dialysate from infusate source 113, as described.

In some embodiments, when the renal care system 100 is in multi-pass mode, or when water is used during single pass mode rather than pre-made dialysate, the dialysate can be regenerated by sorbent cartridge 106, and infusates added from infusate source 113 to regenerate the dialysate. Water can be added from water source 110 or a separate water source if necessary to dilute the dialysate. One or more sensors (not shown) can be included in fluid flow path 101 to ensure that the dialysate entering dialyzer 103 has the proper concentrations of all solutes.

In certain embodiments, the electronic control device(s) 200 can be programmed to receive one or more patient parameters and system parameters and determine a time during a dialysis session to switch the mode of operation between either single pass and multi-pass mode, or by adjusting the water addition rate to add more or less water to renal care system 100. The electronic control device(s) 200 can receive the patient and system parameters through any means. For example, a user interface can be provided for the user to enter the patient and system parameters. Alternatively, the electronic control device(s) 200 can receive the patient and system parameters directly from electronic patient records, from a smart card or similar device associated with the patient or system components, or any other source.

Alternatively, or in addition, a sensor 116 can be included in the fluid flow path 101 that can be used to monitor a dialysis session status and adjust operation when a predetermined clearance level is reached. For example, a urea, creatinine or other sensor 116 between dialyzer outlet 105 and valve 107 in FIG. 1 could detect when the patient has reached a desired percent of clearance in the session. The electronic control device(s) 200 can be programmed to adjust the mode of operation when a predetermined percent of clearance has been reached. An optional sensor 118 downstream of sorbent cartridge 106 can also be included. Sensor 118 can be used to determine if the functional capacity of the sorbent cartridge 106 is exceeded. Sensor 118 can be an ammonia sensor to determine if the adsorptive capacity of the zirconium phosphate has been exceeded. Alternatively, sensor 118 can be a pH sensor to determine if the buffering capacity of zirconium phosphate has been reached. If the capacity of the zirconium phosphate is reached, renal care system 100 can be switched into a single-pass mode.

In some embodiments, the electronic control device(s) 200 may include or be in communication with an image sensor 201. The image sensor 201 may be positioned on, in and/or in proximity to the renal care system 100 such, such as being mounted to a housing 121 of the renal care system. The image sensor 201 may be positioned so as to capture imagery of one or more components of the renal care system 100. In some embodiments, the image sensor 201 may include multiple image sensors 201 to expand a view of the components of the renal care system 100. The image sensor(s) 201 may be positioned to capture imagery of components that are at a greatest risk of fault, e.g., through wear, degradation, corrosion, or other sources of fault or any combination thereof. For example, the image sensor 201 may be positioned and/or oriented towards power electronics (e.g., diodes, electronic switches, amplifiers, power sources, batteries, converters, etc.) and/or the substrate(s) thereof, to control electronics including of the electronic control device(s) 200 among other electronic components and/or the substrate(s) thereof, the valves 107, 109, pumps 102, 111, 114, fluid lines 112, 108, 101, 115, and/or connectors/couplers thereof, among other components or any combination thereof. For example, the power and/or control electronics may include substrates including printed circuit boards on which minerals may be deposited, e.g., as a leak from one or more fluid lines and/or fluid line couplers. Such deposits may be short circuit electronic components, thus causing a fault. As another example, deposits of minerals may build up around joints, corners, etc. leading to reduced and/or blocked fluid flow.

In some embodiments, the electronic control device(s) 200 may receive the imagery of components captured by the image sensor 201 and detect potential faults, occurrences of faults and/or the risk thereof (for simplicity, collectively referred to as “fault risk”) using one or more machine learning algorithms trained to classify such imagery according to fault risk. Based on the contents of the imagery representing the condition of one or more of the components, the machine learning algorithms may output a detected fault risk based on trained machine learning model parameters, such as trained image classification parameters. For example, the machine learning model parameters may be trained to classify images as matching a known fault risk or fault risk type, not matching a known fault risk or fault risk type, matching a known non-fault or low fault risk condition, not matching a known non-fault or low fault risk condition, or any combination thereof.

In some embodiments, based on the detection of a fault risk, the electronic control device(s) 200 may issue a command and/or instruction. Such command and/or instruction may include a device command to cause the renal care system 100 to adjust or terminate operation so as to mitigate the fault. For example, to prevent a short circuit of a power electronic component, the electronic control device(s) 200 may instruct the renal care system 100 to power down. In another example, to protect against a burst fluid line, the electronic control device(s) 200 may instruct a pump to slow or terminate operation when a potential blockage in a fluid line is detected. In some embodiments, fault risks may be mapped to any one or more device commands to adjust the operation of the renal care system 100 to mitigate the fault and effects thereof.

In some embodiments, the electronic control device(s) 200 may issue an alert upon detection of a fault risk. For example, the electronic control device(s) 200 may cause a display device associated with the renal care system 100 to present a prompt indicating that a fault risk is detected and/or a type of fault risk. In another example, the electronic control device(s) 200 may transmit, via a wireless radio and/or wired connection, a communication to another device, such as a computing device of the patient, patient care professional, IT technician or other associated user or any combination thereof.

In some embodiments, the electronic control device(s) 200 may log a detected fault risk, e.g., in a database, memory, error log, cache, or other storage location so as to enable later access and/or tracking of fault risks.

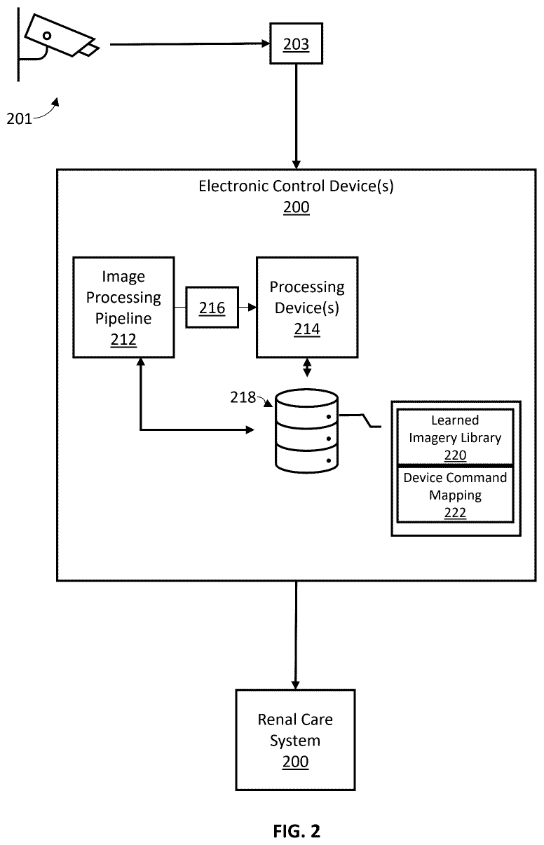

FIG. 2 depicts a block diagram of an exemplary electronic control device(s) configured to detect a state of, and provide electronic instruction to components of the dialysis system in accordance with one or more embodiments of the present disclosure.

In some embodiments, the renal care system 100 may be controlled by the electronic control device(s) 200 that is equipped with one or more image classification machine learning (ML) model(s) of an image processing pipeline 212 to automatically detect, mitigate and/or warn of fault risk to components of the renal care system 100. To do so, the image processing pipeline 212 may include trained model parameters configured to recognize potential fault risks based on learned fault conditions, as well as identify a device command based on the learned fault conditions and/or identify a user to alert upon detection of the fault risk. Where a fault risk is detected, the device command associated with the image may be used to control the renal care system 100 to start, end, adjust, configure or otherwise control a renal care procedure.

In some embodiments, the electronic control device(s) 200 may include hardware components such as one or more processing device(s) 214 and one or more storage device(s) 218. In some embodiments, the processing device(s) 214 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processing device(s) 214 may include data-processing capacity provided by the microprocessor. In some embodiments, the microprocessor may include memory, processing, interface resources, controllers, and counters. In some embodiments, the microprocessor may also include one or more programs stored in memory.

Similarly, the electronic control device(s) 200 may include storage device(s) 218, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage device(s) 218 may include, e.g., a suitable non-transient computer readable medium such as, e.g., random access memory (RAM), read only memory (ROM), one or more buffers and/or caches, among other memory devices or any combination thereof.

In some embodiments, the electronic control device(s) 200 may implement computer engines for implementing the image processing pipeline 212 for image processing and/or classification to detect fault risks. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof.

In some embodiments, to perform fault risk detection and device command generation from images, the electronic control device(s) 200 may include computer engines including, e.g., one or more hardware and/or software components for instantiating and running the image processing pipeline 212. In some embodiments, the image processing pipeline 212 may include dedicated and/or shared software components, hardware components, or a combination thereof. For example, the image processing pipeline 212 may include a dedicated processor and storage. However, in some embodiments, the image processing pipeline 212 may share hardware resources, including the processing device(s) 214 and storage device(s) 218 of the electronic control device(s) 200 via, e.g., a bus.

In some embodiments, the electronic control device(s) 200 may receive an image 203 from the image sensor(s) 201. The image sensor(s) 201 may include one or more image capture devices, such as a digital camera, video camera, smartphone, tablet, or other device having one or more sensors capable of capturing imagery or any combination thereof. In some embodiments, the image sensor(s) 201 may be positioned to capture imagery of a particular component to which the image sensor(s) 201 corresponds, of a particular region or space of the renal care system 100 which includes one or more components thereof, or any combination thereof. There may be multiple image sensor(s) 201, each corresponding to one or more components and/or regions or spaces of the renal care system 100.

In some embodiments, the image sensor(s) 201 may capture images of the renal care system 100 on demand, intermittently and/or continuously. For example, the image sensor(s) 201 may be configured to capture a new image every time the renal care system 100 is turned on, every time a renal care procedure using the renal care system is initiated, every time a renal care procedure using the renal care system is ended, periodically (e.g., every minute, 5 minutes, 10 minutes, 15 minutes, 20 minutes, 25 minutes, 30 minutes, hour, 2 hours, 3 hours, 4 hours, 5 hours, 6 hours, 7 hours, 8 hours, 12 hours, day, week, month, etc.), according to a predetermined servicing schedule, or continuously while the renal care system 100 is on, or any combination thereof.

In some embodiments. In some embodiments, the image sensor(s) 201 may include one or more sensor devices for detecting light in at least one portion of the electromagnetic spectrum (e.g., infrared, visible light, ultraviolet, x-ray, etc.) and producing data forming an image produced by the light. For example, the image sensor(s) 201 may include, e.g., a charge-coupled device (CCD), photodiode, active pixel sensor (APS), complementary metal oxide semiconductor (CMOS), n-type metal oxide semiconductor (NMOS), among other sensors and/or sensor types or any combination thereof.

In some embodiments, the electronic control device(s) 200 may receive the image 203 indirectly from the image sensor(s) 201, such as, e.g., from a storage and/or memory storing the image, such as a memory associated with the controller, an external memory, a remote storage device/system (e.g., cloud storage), or other non-transitory computer readable medium or any combination thereof.

A computer-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

In some embodiments, the image sensor(s) 201 may be a part of the electronic control device(s) 200, the renal care system 100 and/or an external device. The external device may include a user's device, such as, e.g., at least one personal computer (PC), laptop computer, tablet, a mobile phone, smartphone, smart watch, television, smart device (e.g., smart phone, smart tablet or smart television), mobile internet device (MID), messaging device, data communication device, and so forth. The image sensor(s) 201 may integrated into the renal care system 100 as a component thereof, or may be part of an add-on module to be installed on or in the renal care system 100. The add-on module may include one or more processing devices of the electronic control device(s) 200 for implementing the image processing pipeline 212.

In some embodiments, the image sensor(s) 201 may provide the image 203 to the electronic control device(s) 200. In some embodiments, the electronic control device(s) 200 may communicate with the image sensor(s) 201 via a network connection. In some embodiments, the image sensor(s) 201 may interact with the electronic control device(s) 200 using one or more suitable local and/or network communication protocols, such as, e.g., a messaging protocol, a networking protocol, one or more application programming interfaces (APIs), or other suitable technique for communicating between computing systems or any combination thereof. For example, the image sensor(s) 201 may interact with the electronic control device(s) 200 over a network including the Internet to cause the electronic control device(s) 200 to perform automated fault risk detection and control of the renal care system 100. In another example, the electronic control device(s) 200 may be local to the renal care system 100, such as, e.g., a software program installed on the renal care system 100 and configured to employ computing hardware of the renal care system 100 to perform the automated fault risk detection and control of the renal care system 100 In some embodiments, the image 203 may include a representation of the conditions of at least one component of the renal care system 100. The image 203 may be in the form of a raster image, vector image or both, and may be in black and white and/or in color. In some embodiments, the image 203 may be an image file having any suitable image format, such as, e.g., PNG, JPEG, TIFF, PDF, EPS, AVIF, WebP, GIF, EXR, DNG, HEIC, or other suitable image format or any combination thereof and may include raster and/or vector information. In some embodiments, the image 203 may be represented in one or more color spaces according to one or more color models. The color space may include, e.g., sRGB, Adobe RGB, CIELAB, CIEXYZ, Pantone, or other color space or any combination thereof. The color model may include, e.g., RGB, HSL, HSV, CIELAB, CIEXYZ, CMYK, or other color model or any combination thereof.

In some embodiments, the electronic control device(s) 200 may be configured to receive the image 203 to determine whether the image is of a condition in or on one or more components of the renal care system 100 that is indicative of a fault or risk of a future fault. To do so, the electronic control device(s) 200 may employ the image processing pipeline 212 to recognize the fault risk and identify a device command associated with the mitigation of the fault risk. Accordingly, the image processing pipeline 212 may detect that a fault risk and/or recognize a type of fault risk.

In some embodiments, the image processing pipeline 212 may use learned fault imagery to determine whether the image 203 matches to a known fault risk based on a learned imagery library 220 and a device command mapping 222 stored in the storage device(s) 218.

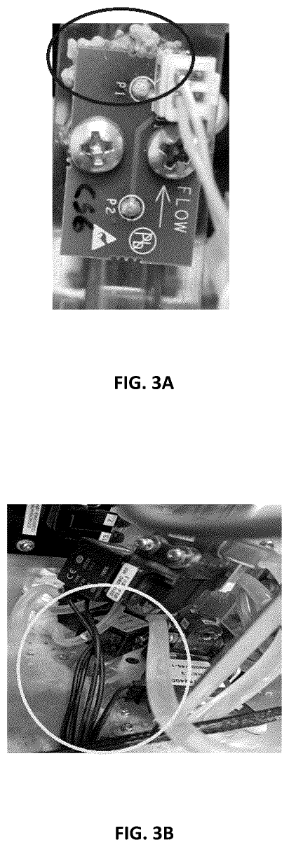

In some embodiments, the learned imagery library 220 may include a library of images acquired through a training stage (see, e.g., FIG. 3) of the image processing pipeline 212. The learned imagery library 220 may include one or more images by an image sensor that represent known fault risks to the components captured in the image(s). The learned imagery of the learned imagery library 220 may include one or more measured and/or derived features associated with the images of the known fault risks as will be described in greater detail below. Thus, the learned imagery may be associated with the conditions of components that are indicative of a fault or risk of fault.

In some embodiments, the learned imagery library 220 may include learned imagery associated with device commands for controlling the renal care system 100. The device commands may be specified in a device command mapping 222 that maps device commands to faults so as to enable automatic retrieval of a device command upon a fault risk being detected, and controlling the renal care system 100 using the retrieved device command so as to mitigate the fault risk. In some embodiments, the device command mapping 222 may include a data structure that maps each device command to a fault risk and/or fault risk type. For example, the image processing pipeline 212 may be configured to determine whether a fault risk is or is not indicated by the conditions captured in the image 203. In such an example, the device commands in the device command mapping 222 may be mapped to the “fault risk” or the “no fault risk” condition. Alternatively or in addition, the image processing pipeline 212 may be configured to recognize a type of fault risk indicated by the condition represented in the image 203. In such a scenario, the one or more of the device commands may be mapped in the device command mapping 222 to a particular fault risk type. Thus, particular device commands can be mapped to particular fault risk types so as to enable quick and efficient determination of a device command that mitigates the fault risk type.

Examples of such device commands may include, e.g., powering the renal care system 100 and/or component(s) thereof on or off, adjusting a flow rate, performing a calibration, initiating cartridge recharging, starting and/or stopping a treatment, defining treatment parameters, clear (e.g., remove, delete, deprecate, mark as resolved, etc.) the errors already stored in the memory after correction, send a treatment report (e.g., by e-mail, in-app notification, API integration, text message, internet message communication, etc.) (such as where the device is connected with ethernet, WiFi, Bluetooth, USB, or other wired and/or wireless connection), enabling/disabling child lock feature, status of the recharge remaining time and therapy remaining time, among other commands or any combination thereof.

In some embodiments, the learned imagery library 220 may include a set of learned imagery for each fault type (e.g., There is a possibility of the system failure during the therapy process due to fluid leakages, electrical sparks, bubbles in tubes, chemical deposition on the connectors of the PCBAs, tube segments, peripheral connectors, valves, corrosion, fluid blockage, mechanical, electrostatic wear, mechanical separation, fracture, tear, etc.). Thus, the calibration process may include training based on one or more training images for each fault type. Thus, the image processing pipeline 212 may be trained to detect a fault risk and/or a fault type based on a new image 203 and the learned imagery.

In some embodiments, the image processing pipeline 212 may include one or more learning models. In some embodiments, machine learning model(s) may include one or more machine learning architectures, including, but not limited to, a decision tree, random forest, an Adaboost, a K Nearest-neighbor, a Support Vector Machine (SVM), a Gaussian Mixture Model (GMM), a Deep Neural Network (DNN), a convolution neural network (CNN), a recurrent neural network (RNN). For example, a DNN approach may facilitate end-to-end speech recognition with or without feature extraction, an RNN approach may facilitate interpreting sequential data such as as follow-up or a series of commands, and a CNN approach may be utilized where the image 203 includes or is used to form a spectrogram and/or spectrogram-based features.

In some embodiments and, optionally, in combination of any embodiment described above or below, an exemplary implementation of Neural Network may be executed as follows:
a. define Neural Network architecture/model,
b. transfer the input data to the exemplary neural network model,
c. train the exemplary model incrementally,
d. determine the accuracy for a specific number of timesteps,
e. apply the exemplary trained model to process the newly-received input data,
f. optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the image processing pipeline 212 may ingest the image 203 and, based on whether a fault risk is detected (and/or a fault type), output a device command 216. In some embodiments, the fault risk may include a percentage indicative of a probability of match to one or more images or one or more faults or fault types in the learned image library 220. Indeed, the image processing pipeline 212 may be trained to determine a match percentage indicative of a probability of a match between the image 203 and each learned image in the learned image library 220. Alternatively, or in addition, the image processing pipeline 212 may learn one or more latent representations of faults and determine the probability of fault in the image 203 based on application of the latent representation. For example, the image processing pipeline 212 can include a neural network configured to perform the fault detection. The neural network may include input data processor, one or more intermediate layer encoding that process the input data through one or more layers to generate intermediate encodings, and output generation to produce an output based on the latent representation formed in the intermediate layers via the training process. In some embodiments, the neural network can be implemented using various architectures, such as convolutional neural networks (CNNs), recurrent neural networks (RNNs), or autoencoders, depending on the specific application requirements.

To do so, in some embodiments, the image processing pipeline 212 may process the image 203 to extract and/or generate one or more features to quantitatively represent the image 203. Such features may be encoded in a feature vector and/or feature map and input into one or more machine learning models, such as those detailed above. In some embodiments, the machine learning model(s) may be trained to detect the fault risks based on the features of the image 203.

In some embodiments, the training may be based on the learned imagery library 220. For example, the features may be used to determine a pattern that represents one or more characteristics of the image 203, and the pattern may be classified based on its similarity to the learned imagery, the similarity being indicative of a likelihood of the image 203 having the same conditions associated with fault risk represented by the image 203. For example, where the similarity measure exceeds a predetermined threshold, the image 203 may be determined to match the learned imagery, and as a result a fault risk may be identifier, e.g., via single class, binary class or multi-class classification. For example, the detection of fault risk may include a single class or binary class classification where the image 203 is classified as including a condition associated with a fault risk or not including/not being able to detect the condition associated with the fault risk. For example, the detection of fault risk may include a multi-class classification wherein the image 203 is classified according to fault risk type. In some embodiments, the image processing pipeline 212 may include two machine learning models, a first binary classifier to determine whether an image includes a condition associated with a fault risk, and thus classify whether a fault risk is detected in the image or not, and then, if a fault risk is detected, a second machine learning model for multi-class classification of the image according to fault risk type.

In some embodiments, one or more machine learning models may be used to generate the features associated with the image 203. Alternatively, or in addition, the features may be generated by one or more statistical models and/or mathematical functions. Alternatively, or in addition, the pattern may be defined by the feature vector encoding the extracted features.

In some embodiments, the image 203 may be classified using one or more machine learning models. The machine learning model(s) may include one or more supervised learning models trained on the learned imagery, such as, but not limited to, an RNN, a CNN, a DNN, a SVM, random forest, decision trees, among others or any combination thereof. The machine learning model(s) may include one or more unsupervised learning models trained on the learned imagery, such as, but not limited to, clustering, association, dimensionality reduction, Gaussian Mixture Model (GMM), autoencoders, among others or any combination thereof. For example, the unsupervised model(s) may include one or more clustering models trained to cluster the features of the image 203 with the learned imagery so as to identify the nearest, and thus most likely, classification.

In some embodiments, whether supervised or unsupervised, the image processing pipeline 212 may output a classification. The classification may include, e.g., the presence or lack of presence of a fault risk, the presence of a fault risk type, the probability or match percentage between the image 203 and one or more faults of one or more fault types represented in the learning image library 220, or any combination thereof. Where the image 203 is confirmed to have a fault risk, the device command may be identified in the device command mapping 222 based on which device command(s) is/are mapped to the fault risk and/or fault risk type. Accordingly, the image processing pipeline 212 may output a device command 216 configured to cause the renal care system 100 to operate according to the device command. Thus, in some embodiments, one or more processing device(s) 214 may instruct the renal care system 200 with the device command 216 so as to control the renal care system 100 and mitigate a fault risk and/or alert a user with a recommendation to respond to the fault risk (e.g., by scheduling maintenance, shutting off the renal care system 100, calling a healthcare provider, replacing a component, etc.).

FIGS. 3A and 3B depict illustrative examples of fault risks for components in a dialysis system in accordance with one or more embodiments of the present disclosure.

In some embodiments, the image 203 may capture the conditions of microcontroller associated with a fluid control component (e.g., a valve, pump or other controllable mechanism) as depicted in FIG. 3A. Such components may be subject to build up of minerals deposited from leaks of a sorbent, dialyzer and/or bodily fluid from nearby components. As a result, the minerals may build-up on the microcontroller, such as on a PCB of the microcontroller substrate, or other portion thereof. Such mineral build-up may pose a risk of a short circuit, corrosion of metals of the microcontroller, or other fault type. Thus, the image processing pipeline 212 may be trained on images of mineral deposits on the fluid control component so as to identify such fault risks in future images.

In some embodiments, the image 203 may capture the conditions of wiring and/or tubing inside the housing 121 of the renal care system 100 as depicted in FIG. 3B. Such components may be subject to leaks of a sorbent, dialyzer and/or bodily fluid. As a result, the components may have a fault risk associated with fluid leakage. Thus, the image processing pipeline 212 may be trained on images of the interior of the housing 121 so as to identify such fault risks, including leaks, in future images.

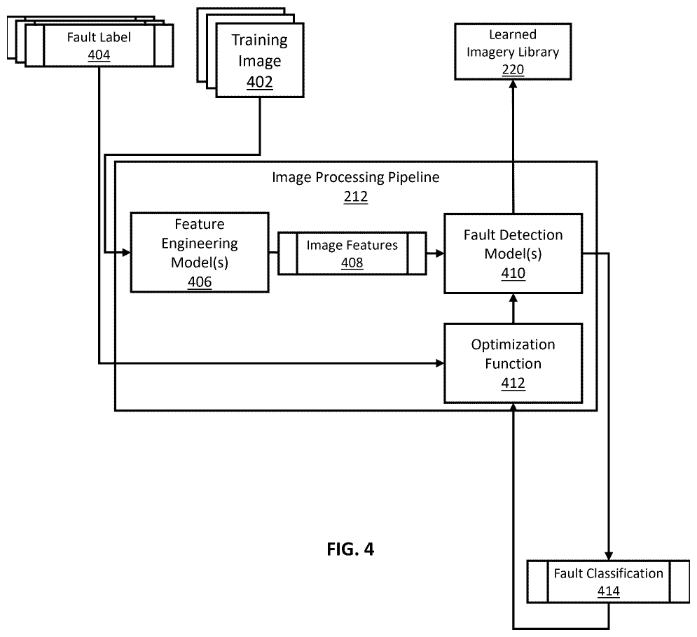

FIG. 4 depicts an exemplary machine learning training methodology for training one or more image classifiers to detect fault risks in a dialysis system in accordance with one or more embodiments of the present disclosure.

In some embodiments, the image processing pipeline 212 may include one or more feature engineering model(s) 406 to pre-process imagery and extract quantitative image features 408, and one or more fault detection model(s) 410 to generate a fault classification 414 predicting whether the imagery depicts a fault condition. In some embodiments, the image processing pipeline 212 may train the fault detection model(s) 410 to output the fault classification 414 by using training data including training images 402 paired with known fault labels 404. The fault labels 404 include ground truth classification data indicative of whether or not a given training image 402 depicts a fault risk. The fault labels 404 may be automatically generated, human annotated, and/or feedback from user acceptance, correction and dispute, e.g., via a user computing device in communication with the electronic control device(s) 200, of a previously predicted fault classification 414.

Accordingly, in some embodiments, the feature engineering model(s) 406 may receive a training image 402 and extract the image features 408 representing the training image 402. In some embodiments, the feature engineering model(s) 406 may include determining one or more pixel values for each pixel in the training image 402, such as by extracting the RGB values for each pixel, the CMYK values for each pixel, the intensity for each pixel, or other values based on the color model and/or color space of the training image 402. In some embodiments, the image features 408 may include the pixels value for each pixel. In some embodiments, the image features 408 may include, instead or in addition, features extracted according one or more other feature extraction techniques, such as histogram of oriented gradients (HOG), Scale Invariant Feature Transform (SIFT), Speeded-Up Robust Feature (SURF), or other technique or any combination thereof.

In some embodiments, HOG analyzes edge orientations within an object represented in the training image 402 to describe its shape and appearance. To do so, HOG may divide the image into small cells, compute gradient orientations within each cell, create histograms of the orientations, and concatenate histograms across cells to form a feature vector representative of objects in the training image 402. Thus, HOG may quantitatively capture shape information for object detection and classification.

In some embodiments, SIFT may detect key points in an image that are invariant to scaling, rotation, and illumination changes. To do so, SIFT may locate key points (also called keypoints) on an object within an image such that the keypoints are robust to changes in scale, rotation, noise, and illumination, such as by identifying high-contrast regions like object edges. In some embodiments, for each keypoint, SIFT computes a descriptor-a quantitative representation of the local feature that encodes information about the keypoints' appearance and orientation. In some embodiments, SIFT may be particular beneficial for improving matching of the training image to learned imagery from the learned imagery library, such as by using clustering and/or similarity measure(s) based fault detection model(s) 410. In some embodiments, the measure of similarity may include, e.g., an exact match or a predetermined similarity score according to, e.g., Jaccard similarity, Jaro-Winkler similarity, Cosine similarity, Euclidean similarity, Overlap similarity, Pearson similarity, Approximate Nearest Neighbors, K-Nearest Neighbors, among other similarity measure. The predetermined similarity score may be any suitable similarity score according to the type of electronic activity to identify a measured attribute of any two data entries as the same.

In some embodiments, similar to SIFT, SURF identifies robust features by analyzing local image regions. To do so, SURF may perform interest point detection, e.g., using integer approximation of Hessian blob detector, and/or computing with 3 integer operations using a precomputed integral image. Based on the interest point detection, SURF may generate a feature description based on the sum of Haar wavelet responses around the point of interest. As with SIFT, SURF may be particular beneficial in improving the performance of clustering and/or similarity based fault detection model(s) 410.

In some embodiments, the feature engineering model(s) 406 may output image features 408, such as pixel values, HOG, SIFT and/or SURF derived feature descriptors, among other features or any combination thereof. In some embodiments, the image features 408 may be encoded in a feature vector and/or feature map to produce a data structure representative of the content so the training image 410 for input into the fault detection model(s) 410.

In some embodiments, the fault detection model(s) 410 may generate the fault classification 414 based on the image features 408 using one or more machine learning and/or statistical/algorithmic models. In some embodiments, the fault detection model(s) 410 ingests a feature vector that encodes features representative of training image 402. In some embodiments, the fault detection model(s) 410 processes the feature vector with parameters to produces a prediction of fault classification 414. In some embodiments, the parameters of the fault detection model(s) 410 may be implemented in a suitable machine learning model including a classifier machine learning model, such as, e.g., a convolutional neural network (CNN), a Naive Bayes classifier, decision trees, random forest, support vector machine (SVM), K-Nearest Neighbors, or any other suitable algorithm for a classification model. In some embodiments, for computational efficiency while preserving accuracy of predictions, the fault detection model(s) 410 may advantageously include a random forest classification model.

In some embodiments, the fault detection model(s) 410 processes the features encoded in the feature vector by applying the parameters of the classifier machine learning model to produce a model output vector. In some embodiments, the model output vector may be decoded to generate one or more labels indicative of fault classification 414. In some embodiments, the model output vector may include or may be decoded to reveal a numerical output, e.g., one or more probability values between 0 and 1 where each probability value indicates a degree of probability that a particular label correctly classifies the training image 402. In some embodiments, the fault detection model(s) 410 may test each probability value against a respective probability threshold. In some embodiments, each probability value has an independently learned and/or configured probability threshold. Alternatively or additionally, in some embodiments, one or more of the probability values of the model output vector may share a common probability threshold. In some embodiments, where a probability value is greater than the corresponding probability threshold, the training image 402 is labeled according to the corresponding label. For example, the probability threshold can be, e.g., greater than 0.5, greater than 0.6, greater than 0.7, greater than 0.8, greater than 0.9, or other suitable threshold value. Therefore, in some embodiments, the fault detection model(s) 410 may produce the fault classification 414 for a particular training image 402 based on the probability value(s) of the model output vector and the probability threshold(s).

In some embodiments, such as in tandem with HOG, SIFT and/or SURF based feature engineering, the fault detection model(s) 410 may employ one or more clustering models that is trained to delineate groups of images based on a similarity measure between the image features 408 of each image. In some embodiments, the measure of similarity may include, e.g., an exact match or a predetermined similarity score according to, e.g., Jaccard similarity, Jaro-Winkler similarity, Cosine similarity, Euclidean similarity, Overlap similarity, Pearson similarity, Approximate Nearest Neighbors, K-Nearest Neighbors, among other similarity measure. The predetermined similarity score may be any suitable similarity score according to the type of electronic activity to identify a measured attribute of any two data entries as the same. In some embodiments, the assignment of the training image 402 to a particular cluster may be dictated based on trained parameters of the clustering model(s) that define the boundary between clusters. Thus, the cluster model may assign the training image 402 to a particular cluster based on the image features 408, and classify the training image 402 based on the cluster assignment.

In some embodiments, the parameters of the fault detection model(s) 410 may be trained based on fault label 404s. For example, the training image 402 may be paired with a target classification or known classification to form a training pair, such as a historical training image 402 and an observed result and/or human annotated classification denoting whether the historical training image 402 is indicative of a fault risk and/or fault risk type. In some embodiments, the training image 402 may be provided to the fault detection model(s) 410, e.g., encoded in a feature vector, to produce a predicted label. In some embodiments, an optimization function 412 associated with the fault detection model(s) 410 may then compare the predicted label with the fault label 404 of a training pair including the historical training image 402 to determine an error of the predicted label. In some embodiments, the optimization function 412 may employ a loss function, such as, e.g., Hinge Loss, Multi-class SVM Loss, Cross Entropy Loss, Negative Log Likelihood, or other suitable classification loss function to determine the error of the predicted label based on the fault label 404.

In some embodiments, the fault label 404 may be obtained after the fault detection model(s) 410 produces the prediction, such as in online learning scenarios. In such a scenario, the fault detection model(s) 410 may receive the training image 402 and generate the model output vector to produce a label classifying the training image 402. Subsequently, a user may provide feedback by, e.g., modifying, adjusting, removing, and/or verifying the label via a suitable feedback mechanism, such as a user interface device (e.g., keyboard, mouse, touch screen, user interface, or other interface mechanism of a user device or any suitable combination thereof). The feedback may be paired with the training image 402 to form the training pair and the optimization function 412 may determine an error of the predicted label using the feedback.

In some embodiments, based on the error, the optimization function 412 may update the parameters of the fault detection model(s) 410 using a suitable training algorithm such as, e.g., backpropagation for a classifier machine learning model. In some embodiments, backpropagation may include any suitable minimization algorithm such as a gradient method of the loss function with respect to the weights of the classifier machine learning model. Examples of suitable gradient methods include, e.g., stochastic gradient descent, batch gradient descent, mini-batch gradient descent, or other suitable gradient descent technique. As a result, the optimization function 412 may update the parameters of the fault detection model(s) 410 based on the error of predicted labels in order to train the fault detection model(s) 410 to model the correlation between training image 402 and fault classification 414 in order to produce more accurate labels of training image 402.

In some embodiments, the training image 402 and corresponding fault label 404 may be added to the learned imagery library 220, e.g., as part of a training dataset and/or for clustering in future classification tasks.

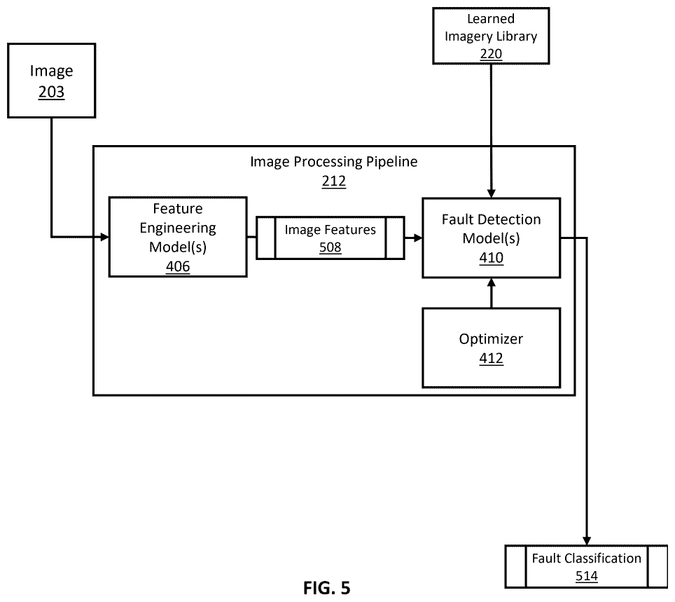

FIG. 5 depicts an exemplary machine learning classification methodology for detecting fault risks in a dialysis system in accordance with one or more embodiments of the present disclosure.

In some embodiments, the image processing pipeline 212 may include one or more feature engineering model(s) 406 to pre-process imagery and extract quantitative image features 508, and one or more fault detection model(s) 410 to generate a fault classification 514 predicting whether the imagery depicts a fault condition. In some embodiments, the image processing pipeline 212 may include the fault detection model(s) 410 having parameters training using supervised and/or unsupervised training, e.g., as detailed above such as in reference to FIG. 4.

Accordingly, in some embodiments, the feature engineering model(s) 406 may receive the image 203 and extract the image features 508 representing the image 203. In some embodiments, the feature engineering model(s) 406 may include determining one or more pixel values for each pixel in the image 203, such as by extracting the RGB values for each pixel, the CMYK values for each pixel, the intensity for each pixel, or other values based on the color model and/or color space of the image 203. In some embodiments, the image features 508 may include the pixels value for each pixel. In some embodiments, the image features 508 may include, instead or in addition, features extracted according one or more other feature extraction techniques, such as histogram of oriented gradients (HOG), Scale Invariant Feature Transform (SIFT), Speeded-Up Robust Feature (SURF), or other technique or any combination thereof as detailed above to produce image features 508, such as pixel values, HOG, SIFT and/or SURF derived feature descriptors, among other features or any combination thereof. In some embodiments, the image features 508 may be encoded in a feature vector and/or feature map to produce a data structure representative of the content so the training image 410 for input into the fault detection model(s) 410.

In some embodiments, the fault detection model(s) 410 may generate the fault classification 514 based on the image features 508 using one or more machine learning and/or statistical/algorithmic models. In some embodiments, the fault detection model(s) 410 ingests a feature vector that encodes features representative of image 203. In some embodiments, the fault detection model(s) 410 processes the feature vector with parameters to produces a prediction of fault classification 514. In some embodiments, the parameters of the fault detection model(s) 410 may be implemented in a suitable machine learning model including a classifier machine learning model, such as, e.g., a convolutional neural network (CNN), a Naive Bayes classifier, decision trees, random forest, support vector machine (SVM), K-Nearest Neighbors, or any other suitable algorithm for a classification model. In some embodiments, for computational efficiency while preserving accuracy of predictions, the fault detection model(s) 410 may advantageously include a random forest classification model.

In some embodiments, the fault detection model(s) 410 processes the features encoded in the feature vector by applying the parameters of the classifier machine learning model to produce a model output vector. In some embodiments, the model output vector may be decoded to generate one or more labels indicative of fault classification 514. In some embodiments, the model output vector may include or may be decoded to reveal a numerical output, e.g., one or more probability values between 0 and 1 where each probability value indicates a degree of probability that a particular label correctly classifies the image 203. In some embodiments, the fault detection model(s) 410 may test each probability value against a respective probability threshold. In some embodiments, each probability value has an independently learned and/or configured probability threshold. Alternatively or additionally, in some embodiments, one or more of the probability values of the model output vector may share a common probability threshold. In some embodiments, where a probability value is greater than the corresponding probability threshold, the image 203 is labeled according to the corresponding label. For example, the probability threshold can be, e.g., greater than 0.5, greater than 0.6, greater than 0.7, greater than 0.8, greater than 0.9, or other suitable threshold value. Therefore, in some embodiments, the fault detection model(s) 410 may produce the fault classification 514 for a particular image 203 based on the probability value(s) of the model output vector and the probability threshold(s).

In some embodiments, such as in tandem with HOG, SIFT and/or SURF based feature engineering, the fault detection model(s) 410 may employ one or more clustering models that is trained to delineate groups of images based on a similarity measure between the image features 508 of each image. For example, the clustering model may output the fault classification 514 for the image 203 based on clustering the image features 508 of the image 203 with, e.g., of learned imagery accessed in the learned imagery library 220. In some embodiments, the measure of similarity may include, e.g., an exact match or a predetermined similarity score according to, e.g., Jaccard similarity, Jaro-Winkler similarity, Cosine similarity, Euclidean similarity, Overlap similarity, Pearson similarity, Approximate Nearest Neighbors, K-Nearest Neighbors, among other similarity measure. The predetermined similarity score may be any suitable similarity score according to the type of electronic activity to identify a measured attribute of any two data entries as the same. In some embodiments, the assignment of the image 203 to a particular cluster may be dictated based on trained parameters of the clustering model(s) that define the boundary between clusters. Thus, the cluster model may assign the image 203 to a particular cluster based on the image features 508, and classify the image 203 based on the cluster assignment.

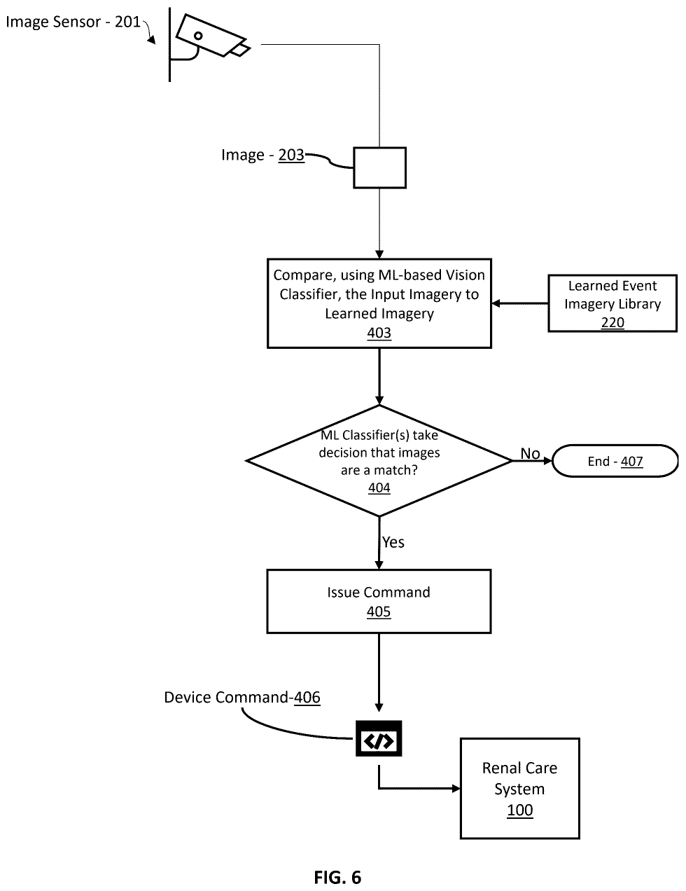

FIG. 6 is a flow chart for a method of detecting fault risks in a dialysis system in accordance with one or more embodiments of the present disclosure.

In some embodiments, an image sensor 201 may produce an image 203 of one or more components of a renal care system 100. In some embodiments, the image 203 may be of a particular component, a group of components, an interior of the housing 121 of the renal care system 100, an exterior of the renal care system 100, or any combination thereof. Thus, the image 203 may capture the component(s), including the conditions thereof at a particular time.

In some embodiments, at block 403, the image may be input into a fault risk detection machine learning model to output at least one detected fault risk in the at least one component based at least in part on trained machine learning model parameters. In some embodiments, as detailed above, the trained machine learning model parameters are configured, through training, to classify the at least one detected fault risk as matching to at least one known fault risk, e.g., in the learned event imagery library 220. Thus, the fault risk detection machine learning model may include an ML-based vision classifier that compares the image 203 to learned imagery, either explicitly, e.g., through clustering and/or similarity measurement, or indirectly via one or more trained neural networks or other ML-based classifiers or any combination thereof, as detailed above.

In some embodiments, upon comparing the image 203 to the learned imagery, whether the ML classifier(s) determines that the image 203 is a match to learned imagery is tested at 404. If there is not match, then the component(s) are presumed to be in operational condition, and the process ends at 407.

Where the ML classifier(s) take the decision that the image 203 matches to learned imagery associated with a fault risk, a command may be issued at 405, thus outputting device command 406 to the renal care system 200. In some embodiments, the match to learned imagery may include a classification of the image 203 that there is a detected fault risk including whether a fault risk exists or not. In some embodiments, the classification may, also or instead, include a type of fault risk present in the component(s) represented in the image 203, such as a corrosion fault risk, leak fault risk, mechanical wear fault risk, or other mechanical, electrical, chemical, etc. fault risk or any combination thereof.

In some embodiments, the determination at 404 may also include generating and/or transmitting an alert upon the detection of a fault risk. For example, an alert may be generated and communicated to a service endpoint associated with one or more service technicians, facilities and/or entities that may perform service on the renal care system 100. Thus, embodiments herein may automatically control the renal care system 100 to mitigate fault risks and/or automatically alert personnel to potential faults in the renal care system 100 for maintenance, servicing and/or repair.

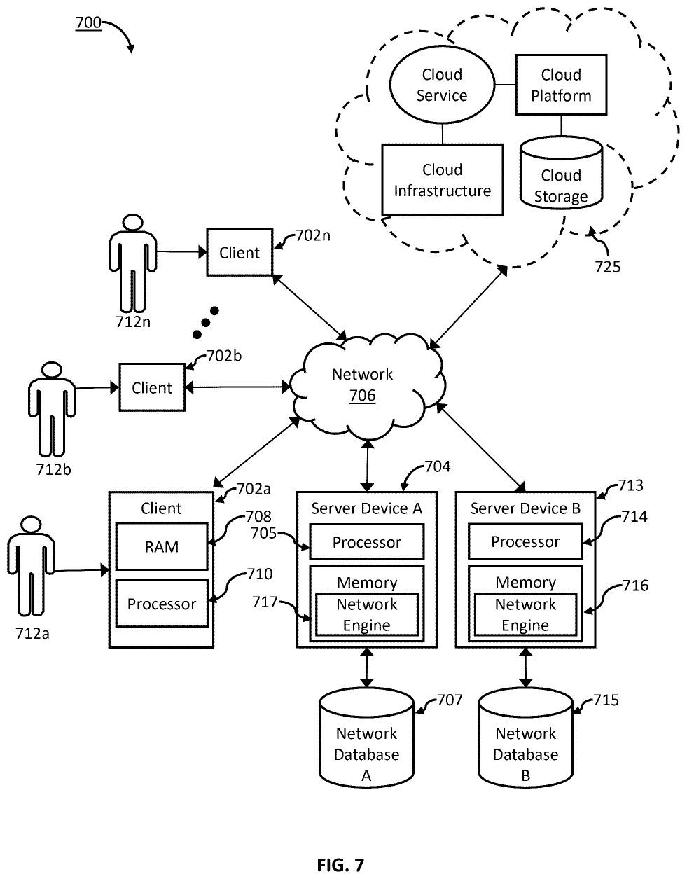

FIG. 7 depicts a block diagram of an exemplary computer-based system and platform 700 for fault risk detection in a dialysis system in accordance with one or more embodiments of the present disclosure. However, not all of these components may be required to practice one or more embodiments, and variations in the arrangement and type of the components may be made without departing from the spirit or scope of various embodiments of the present disclosure.

In some embodiments, the electronic control device(s) 200 and/or the renal care system 100 may client devices on a network 706 so as to be networked with other client devices, e.g., for remote control, monitoring, updating, maintenance, and/or administration. The network 706 may include the client device 702a, client device 702b through client device 702n, any one or more of which may include the electronic control device(s) 200 and/or the renal care system 100, among other computing devices associated with patients, patient care professionals, information technology (IT) technicians, among other users or any combination thereof.

As shown, each of the client device 702a, client device 702b through client device 702n may include a computer-readable medium, such as a random-access memory (RAM) 708 coupled to a processor 710 or FLASH memory. In some embodiments, the processor 710 may execute computer-executable program instructions stored in memory 708. In some embodiments, the processor 710 may include a microprocessor, an ASIC, and/or a state machine. In some embodiments, the processor 710 may include, or may be in communication with, media, for example computer-readable media, which stores instructions that, when executed by the processor 710, may cause the processor 710 to perform one or more steps described herein. In some embodiments, examples of computer-readable media may include, but are not limited to, an electronic, optical, magnetic, or other storage or transmission device capable of providing a processor, such as the processor 710 of client device 702a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, client devices 702a through 702n may also comprise a number of external or internal devices such as a mouse, a CD-ROM, DVD, a physical or virtual keyboard, a display, or other input or output devices. In some embodiments, examples of client devices 702a through 702n (e.g., clients) may be any type of processor-based platforms that are connected to a network 706 such as, without limitation, personal computers, digital assistants, personal digital assistants, smart phones, pagers, digital tablets, laptop computers, Internet appliances, and other processor-based devices. In some embodiments, client devices 702a through 702n may be specifically programmed with one or more application programs in accordance with one or more principles/methodologies detailed herein. In some embodiments, client devices 702a through 702n may operate on any operating system capable of supporting a browser or browser-enabled application, such as MicrosoftTM, Windows™, and/or Linux. In some embodiments, client devices 702a through 702n shown may include, for example, personal computers executing a browser application program such as Microsoft Corporation's Internet Explorer™M, Apple Computer, Inc.'s SafariTM, Mozilla Firefox, and/or Opera. In some embodiments, through the member computing client devices 702a through 702n, user 712a, user 712b through user 712n, may communicate over the exemplary network 706 with each other and/or with other systems and/or devices coupled to the network 706. As shown in FIG. 7, exemplary server devices 704 and 713 may include processor 705 and processor 714, respectively, as well as memory 717 and memory 716, respectively. In some embodiments, the server devices 704 and 713 may be also coupled to the network 706. In some embodiments, one or more client devices 702a through 702n may be mobile clients.

In some embodiments, at least one database of exemplary databases 707 and 715 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method comprising:
receiving, by at least one processor from at least one image sensor positioned on a renal care device, at least one image of at least one component of the renal care device;
inputting, by the at least one processor, the at least one image into a fault risk detection machine learning model to output at least one detected fault risk in the at least one component based at least in part on trained machine learning model parameters;
wherein the trained machine learning model parameters are configured, through training, to classify the at least one detected fault risk as matching to at least one known fault risk;
wherein the training comprises:
inputting a plurality of training images into the fault risk detection machine learning model, the plurality of training images comprising known fault risks to the at least one component of the renal care device, and
updating a plurality of machine learning model parameters based at least in part on the plurality of training images and the known fault risks so as to produce the trained machine learning model parameters;
generating, by the at least one processor, at least one alert to at least one operator of the renal care device, the at least one alert comprising an indication of the at least one detected fault risk; and
controlling, by the at least one processor, the renal care device to terminate operation of the at least one component in response to the at least one detected fault risk.

2. The method of claim 1, further comprising generating, by the at least one processor, at least one control signal to the renal care device based at least in part on the at least one detected fault risk, the at least one control signal being configured to modify operation of the renal care device to mitigate the at least one detected fault risk.

3. The method of claim 2, wherein the at least one control signal comprising a power control signal configured to switch the at least one component off.

4. The method of claim 1, wherein the fault risk detection machine learning model comprises at least one clustering algorithm.

5. The method of claim 1, wherein the fault risk detection machine learning model comprises at least one neural network classifier.

6. The method of claim 1, wherein the at least one detected fault risk comprises at least one fault risk classification identifying a type of fault risk.

7. The method of claim 6, wherein the at least one fault risk classification comprises condensation on the at least one component.

8. The method of claim 6, wherein the at least one fault risk classification comprises corrosion on the at least one component.

9. The method of claim 1, further comprising transmitting, by the at least one processor, the at least one alert to at least one service technician, the at least one alert comprising renal care device data for identifying the renal care device for service.

10. The method of claim 1, wherein the fault risk detection machine learning model is configured to determine a similarity measure between the at least one image and each training image of the plurality of training images, and output the at least one detected fault risk where the similarity measure exceeds a predetermined threshold.

11. A system comprising:
a renal care device comprising at least one component;
at least one image sensor, and
at least one processor, wherein the at least one processor is configured to perform steps to:
receive, from at least one image sensor positioned on a renal care device, at least one image of at least one component of the renal care device;
input the at least one image into a fault risk detection machine learning model to output at least one detected fault risk in the at least one component based at least in part on trained machine learning model parameters;
wherein the trained machine learning model parameters are configured, through training, to classify the at least one detected fault risk as matching to at least one known fault risk;
wherein the training comprises:
inputting a plurality of training images into the fault risk detection machine learning model, the plurality of training images comprising known fault risks to the at least one component of the renal care device, and
updating a plurality of machine learning model parameters based at least in part on the plurality of training images and the known fault risks so as to produce the trained machine learning model parameters;
generate at least one alert to at least one operator of the renal care device, the at least one alert comprising an indication of the at least one detected fault risk; and
control the renal care device to terminate operation of the at least one component in response to the at least one detected fault risk.

12. The system of claim 11, wherein the at least one processor is further configured to perform steps to generate at least one control signal to the renal care device based at least in part on the at least one detected fault risk, the at least one control signal being configured to modify operation of the renal care device to mitigate the at least one detected fault risk.

13. The system of claim 12, wherein the at least one control signal comprising a power control signal configured to switch the at least one component off.

14. The system of claim 11, wherein the fault risk detection machine learning model comprises at least one clustering algorithm.

15. The system of claim 11, wherein the fault risk detection machine learning model comprises at least one neural network classifier.

16. The system of claim 11, wherein the at least one detected fault risk comprises at least one fault risk classification identifying a type of fault risk.

17. The system of claim 16, wherein the at least one fault risk classification comprises condensation on the at least one component.

18. The system of claim 16, wherein the at least one fault risk classification comprises corrosion on the at least one component.

19. The system of claim 11, wherein the at least one processor is further configured to perform steps to transmit the at least one alert to at least one service technician, the at least one alert comprising renal care device data for identifying the renal care device for service.

20. The system of claim 11, wherein the fault risk detection machine learning model is configured to determine a similarity measure between the at least one image and each training image of the plurality of training images, and output the at least one detected fault risk where the similarity measure exceeds a predetermined threshold.
